SEQUENCE LISTING (7 sequences)

SEQ ID NO: 1 - DNA, 925 bp, Pichia pastoris
tcctcacaggtaaggtagaattaccagaaagagaggaacaagaggcttggattcagaacagagtcaatctgaagtctgacaccacctttttccacatgattccatacgaagaggctggtgaatatttccaggacttggtcaggctggctgctgatcctgaattgacttttgacagcgacaaaatctacaacgctaccaaagcaggccaagaactcaagtcgaaatactgggcccgtaacaaagagatcagaaaattgggaaccagtgcttaatccatacgtcgctggcattattaaactaggttttgatgaaaactcagcatgtgattgagaactctcagttggttctttttcgttttcactttacgaattgttttagaaaggcatcatctaatgtaatctgtatacactttattgcattatatatactccaccttgctgttcatctcgtcatcttcggattcttagctgctccatctgccccgggggtgtgaaatgtccgactctccgagcagacggttgagcctcgcgccgcatcaacggataaggcatggtagtgacccctctcaacagcgggccatacagttctccgcccaccgtcgccgcaaggatcaccataaaaccttgctgctcccggcgttcttgggtacttgacggacggagaacagacaggtttcagacccccccggaagttgacacctaggtcagttataattgcaagtcacactacctgcacatagtaaattgctctcacccggttgagatccgaccagttctgttgactctgtttgcttccatgactctgctccccttggccgactgataagcatgttcatcccatcggccattgccgtgactcgggaatgacaccccagcaaacgtatataaacccacaatccccccagatttaccgctttatcacaaaa

SEQ ID NO: 2 - DNA, 30 bp, Artificial Sequence
  primer
agatcttcctcacaggtaaggtagattacc

SEQ ID NO: 3 - DNA, 31 bp, Artificial Sequence
  primer
gaattcttttgtgataaagcggtaaatctgg

SEQ ID NO: 4 - DNA, 642 bp, Pichia pastoris
gtttgttgtcttttataaatgtctttttatatatgcattcatgccttgacagcattgtttggttaatttcgggtcaattgagttcgaatatatcattcgatgtaagtcttgcacgaatgactgtagcacaagatgatgctccacgatggaatcgattctggcgtaggggacgttcaaatctaccagcatctgtttgacaaaattttgaagtctcctaagagagagattgtctactacagcaaggccgagttcattgtatttatcgccttcgaagtcacttttgttcagagatcgaactttacctattcttgttgccaactggtggactagcttcgagttgagtgtctttgattcttgcaattgttgatttatctttgctattttgctgtctttttctgtggattgttcctttaatgtgaatattagtgagtctttgtggtctatcatttcttttagtctgcgaatttgctcgtctttatcatctgtctgtgtttcgtgttcgccttcttttatcgtcatagagctgtcatgagtacctggaggtgttaatagattggtggtggaaccgttatctccttcttcactcacatcgctgaccagttctgctatacgctgtcccgggttgttatggctttttggttc

SEQ ID NO: 5 - DNA, 63 bp, Artificial Sequence
  multiple cloning site
gacgtcagatctcggaattcagcgctggtacctctagactgcaggcggccgcgggatccatgt

SEQ ID NO: 6 - DNA, 31 bp, Artificial Sequence
  primer
gcggccgcgtttgttgtcttttataaatgtc

SEQ ID NO: 7 - DNA, 35 bp, Artificial Sequence
  primer
ggatccaacacgaaacacagacagatgataaagac

(19) United States
(12) Patent Application Publication
(10) Pub. No.: US 2008/0299616 A1
(43) Pub. Date: Dec. 4, 2008

(54) Malate Synthase Regulatory Sequences for Heterologous Gene Expression in Pichia

(76) Inventor: Byung-Kwon Choi, Norwich, VT (US)

Correspondence Address: MERCK AND CO., INC, P O BOX 2000, RAHWAY, NJ, 07065-0907, US

(21) Appl. No.: 11/658,113

(22) PCT Filed: Jul. 22, 2005
(86) PCT No.: PCT/IB05/52479

§ 371 Date: Jun. 4, 2008

Related U.S. Application Data
(60) Provisional application No. 60/590,756, filed on Jul. 22, 2004.

(51) Int. Cl. C12P 21/04; C12N 15/11; C12N 15/00; C12N 1/19

(52) U.S. Cl. 435/69.2; 536/23.2; 435/320.1; 435/254.23; 435/69.1; 435/69.4; 435/69.51; 435/69.6

(57) ABSTRACT

The present invention relates to the isolation of DNA regulatory regions of the malate synthase 1 gene (MLS1) from the yeast, Pichia pastoris. The present invention further relates to the expression of heterologous proteins which have been introduced into a P. pastoris host cell and can be expressed under the regulation of the MLS1 5′ regulatory promoter and MLS1 3′regulatory terminator regions. The MLS1 promoter is repressed in media containing glucose and derepressed under glucose starvation conditions or when acetate is present.

### CROSS-REFERENCE TO RELATED APPLICATIONS

This application claims priority to U.S. provisional application Ser. No. 60/590,756 filed on Jul. 22, 2004, which is incorporated by reference herein in its entirety.

### FIELD OF THE INVENTION

The present invention relates to the isolation of polynucleotides comprising regulatory nucleotide sequences, vectors, and expression cassettes containing such regulatory sequences, and host cells comprising the vectors and/or expression cassettes. In particular, the invention relates to 5′ upstream regulatory nucleotide sequences within the promoter and 3′ downstream regulatory nucleotide sequences within the terminator regions of a malate synthase gene, vectors and expression cassettes containing such regulatory sequences, and host cells comprising the vectors and/or expression cassettes.

### Disclosure of Invention

### BACKGROUND OF THE INVENTION

The methylotrophic yeast Pichia pastoris has been used extensively for re-combinant protein expression. Most Pichia expression vectors are quite similar and are designed to use the promoter from the alcohol oxidase gene (AOX1). This methanol-induced promoter is efficient and tightly regulated, and has been used successfully to induce the expression of heterologous proteins for several years (Cereghino and Cregg, 2000).

Despite its advantages, the AOXI promoter is not ideal for scaled-up expression of proteins, as the amount of methanol required poses engineering and safety challenges—e.g. the volatility and explosion risks associated with large quantities of methanol are not trivial for any laboratory. Furthermore, the addition of methanol not only induces the expression of the desired recombinant protein, but also the protein encoded by the AOX1 gene. This results in high levels of alcohol oxidase which creates a metabolic burden, and can interfere with cell function and analysis (Zupan et al., 2004). Thus, it is desirable to have efficient and tightly regulated promoters for the expression of re-combinant proteins in P. pastoris.

Isocitrate lyase and malate synthase are the key enzymes of the glyoxylate cycle, and are required for the proliferation of yeast cells on C2 substrates such as ethanol or acetate. Specifically, one of the malate synthase genes, MLS1, is highly transcribed on nonfermentable carbon sources and is essential for cell growth on these media. The MLS1 promoter when fused to a reporter protein in Saccaromyces cerevisiae, displays basal level expression in the presence of 2% glucose, and is derepressed more than 100-fold under conditions of sugar limitations (Caspary et al., 1997). Furthermore, it is possible to decouple the carbon source metabolism from induction of the recombinant protein, as a media of both glucose and acetate will provide the preferred glucose for the host cell and the required acetate for heterologous protein induction.

What is desired, therefore, is a promoter for regulated expression of recombinant heterologous proteins in the yeast species, Pichia.

### SUMMARY OF THE INVENTION

The present invention provides an isolated polynucleotide comprising a regulatory region containing a nucleotide sequence of the non-coding region of the MLS1 gene in Pichia pastoris. The isolated polynucleotide of the present invention comprises or consists of a nucleic acid sequence selected from the group consisting of: (a) SEQ ID NO: 1; (b) the complement of SEQ ID NO: 1;

(c) a nucleic acid sequence at least 70%, at least 75%, at least 80%, at least 85%, at least 90%, at least 95%, at least 98%, at least 99% or at least 99.9% (similarity) (idenity) to SEQ ID NO: 1; and (d) a nucleic acid sequence that hybridizes under stringent conditions to SEQ ID NO: 1.

The present invention provides an isolated polynucleotide comprising a regulatory region containing a nucleotide sequence of the non-coding region of the MLS1 gene in Pichia pastoris. The isolated polynucleotide of the present invention comprises or consists of a nucleic acid sequence selected from the group consisting of: (a) SEQ ID NO:4; (b) the complement of SEQ ID NO: 4;

(c) a nucleic acid sequence at least 60%, at least 75%, at least 80%, at least 85%, at least 90%, at least 95%, at least 98%, at least 99% or at least 99.9% (similarity) (identity) to SEQ ID NO: 4; and (d) a nucleic acid sequence that hybridizes under stringent conditions to SEQ ID NO: 4.

The present invention further provides a method for producing heterologous proteins in a host from the genus of Pichia comprising the step of

(a) introducing into the host the polynucleotide sequence of claim 1 or 2; and

(b) culturing the host to produce the heterologous protein.

### BRIEF DESCRIPTION OF THE DRAWINGS

FIG. 1A: shows the pUC19 vector used as described in Example 2. FIG. 1B. shows the expression vector, pBK289 as described in Example 2. FIG. 1C. shows pBK342.5 as described in Example 2. FIG. 1D. shows pBK381 as described in Example 2. FIG. 1E. shows pBK396 as described in Example 2. FIG. 1F. shows pBK398 as described in Example 2.

FIG. 2: depicts a Coomassie stained SDS-PAGE gel of culture supernatant from strain BKY285 Lane 1: molecular weight marker (MW); Lane 2: glucose-repressed (‘uninduced’) culture supernatant and Lane 3: glucose starved (‘induced’) culture supernatant, showing the induction of human plasminogen Kringle 3 reporter protein under control of the P. pastoris inducible MLS1 promoter (as described in Example 4).

### BRIEF DESCRIPTION OF THE SEQUENCES

SEQ ID NO: 1 P. pastoris MLS1 promoter

SEQ ID NO: 2 PpMLS1-P/UP primer

SEQ ID NO: 3 PpMLS1-P/LP primer:

SEQ ID NO: 4 P. pastoris MLS1 transcriptional terminator

SEQ ID NO: 5 PpMLS1-TT/UP primer

SEQ ID NO: 6 PpMLS1-TT/LP primer.

SEQ ID NO: 7 multiple cloning site (MCS)

### DETAILS OF THE INVENTION

### Definitions

Terms and abbreviations as used herein are defined below:

As used herein, ‘isolated polynucleotide’ means a polynucleotide that is free of one or both of the nucleotide sequences which flank the polynucleotide in the naturally-occurring genome of the organism from which the polynucleotide is derived. The term includes, for example, a polynucleotide or variant thereof that is incorporated in a vector or expression cassette; into an autonomously replicating plasmid or virus; into the genomic DNA of a prokaryote or eukaryote; or that exists as a separate molecule independent of other polynucleotides. It also includes a recombinant chimeric polynucleotide that is part of a hybrid polynucleotide, for example one encoding a polypeptide sequence. ‘Isolated polynucleotide’ does not necessarily mean a polynucleotide that has been physical removed from any flanking DNA sequences.

As used herein, ‘polynucleotide’ and ‘oligonucleotide’ are used interchangeably and refer to a polymeric (2 or more monomers) form of nucleotides of any length, either ribonucleotides or deoxyribonucleotides. Although nucleotides are usually joined by phosphodiester linkages, the term also includes polymeric nucleotides containing neutral amide backbone linkages composed of aminoethyl glycine units. This term refers only to the primary structure of the molecule. Thus, this term includes double- and single-stranded DNA and RNA. It also includes known types of modifications, for example, labels, methylation, ‘caps’, substitution of one or more of the naturally occurring nucleotides with an analog, internucleotide modifications such as, for example, those with uncharged linkages (e.g., methyl phosphonates, phosphotriesters, phosphoamidates, carbamates, etc.), those containing pendant moieties, such as, for example, proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g. alpha anomeric nucleic acids, etc.), as well as unmodified forms of the polynucleotide. Polynucleotides include both sense and antisense strands.

As used herein, ‘sequence’ means the linear order in which monomers occur in a polymer, for example, the order of amino acids in a polypeptide or the order of nucleotides in a polynucleotide.

As used herein, ‘variant’ of a reference nucleic acid sequence encompasses nucleic acid sequences that are not necessarily identical in sequence but that are homologous to one another within one or more particular segments. The term ‘degenerate oligonucleotide’ or ‘degenerate primer’ is used to signify an oligonucleotide capable of hybridizing with target nucleic acid sequences that are not necessarily identical in sequence but that are homologous to one another within one or more particular segments.

As used herein, the term ‘complementary’ or ‘complement’ refer to the pairing of bases, purines and pyrimidines that associate through hydrogen bonding in double stranded nucleic acid. The following base pairs are complementary: guanine and cytosine; adenine and thymidine; and adenine and uracil. The terms as used herein include complete and partial complementarity.

As used herein, the term ‘hybridization’ refers to a process in which a strand of nucleic acid joins with a complementary strand through base pairing. The conditions employed in the hybridization of two non-identical, but very similar, complementary nucleic acids vary with the degree of complementarity of the two strands and the length of the strands. Thus the term contemplates partial as well as complete hybridization. Such techniques and conditions are well known to practitioners in this field.

As used herein, ‘expression cassette’ means a genetic module comprising a gene and the regulatory regions necessary for its expression, which may be incorporated into a vector.

As used herein, ‘operably linked’ means any linkage, irrespective of orientation or distance, between a regulatory sequence and coding sequence, where the linkage permits the regulatory sequence to control expression of the coding sequence.

As used herein, ‘heterologous DNA coding sequence’ means any coding sequence other than the one that naturally encodes the malate synthase protein, or any homolog of the malate synthase protein.

As used herein, ‘regulatory sequence’ or ‘regulatory region’ as used in reference to a specific gene, refers to the coding or non-coding nucleotide sequences within that gene that are necessary or sufficient to provide for the regulated expression of the coding region of a gene. Thus, the term encompasses promoter sequences, regulatory protein binding sites, upstream activator sequences and the like. Specific nucleotides within a regulatory region may serve multiple functions. For example, a specific nucleotide may be part of a promoter and participate in the binding of a transcriptional activator protein.

As used herein, ‘coding region’ refers to that portion of a gene which codes for a protein. The term ‘non-coding region’ refers to that portion of a gene that is not a coding region.

Isolation and Induction of the Pichia pastoris MLS1 Promoter

The Pichia expression vectors presently available are designed to use the promoter from the alcohol oxidase gene (AOX1). In addition to the large methanol requirement for AOX1 induction, which poses engineering and safety challenges, the shift from glucose to methanol not only creates a metabolic burden from the induction of large quantities of the Aox1 protein, but this shift in carbon source often results in cell lysis.

The MLS1 promoter region has not been previously cloned from the P. pastoris genome and has not previously been shown to promote protein expression of heterologous proteins in P. pastoris. The isolation of this promoter and terminator sequence as described herein, allows for regulation of heterologous proteins in Pichia with the possibility of decoupling the carbon source metabolism from the induction of the heterologous protein, as a media of both glucose and acetate will provide the preferred glucose for the host cell and the required acetate for heterologous induction.

The present invention provides regulatory nucleotide sequences that are part of the non-coding region of the MLS1 gene in Pichia pastoris. In one embodiment, the 5′ regulatory region of the MLS1 gene, is an inducible promoter, and preferably is induced by ethanol or acetate, and can also be derepressed under glucose starvation conditions.

In one aspect of the present invention, the MLS1 promoter is cloned from a wild type P. pastoris strain, NRRLY-11430 (American Type Culture Collection, ATCC) as a 925 nucleotide fragment corresponding to the 5′ regulatory non-coding region region (SEQ ID NO: 1) directly upstream of the MLS1 gene using a 5′ primer (PpMLS1-P/UP) (SEQ ID NO: 2) and a 3′ primer (PpMLS1-P/LP) (SEQ ID NO: 3) (Example 1). The isolated fragment is subsequently sequenced using the same cloning primers confirming the MLS1 promoter sequence.

The present invention encompasses an isolated polynucleotide comprising a regulatory region containing SEQ ID NO: 1, a variant of SEQ ID NO: 1, the complement of SEQ ID NO: 1, or a variant of the complement of SEQ ID NO: 1. In one embodiment, the polynucleotide (or a variant, or the complement of either the polynucleotide or the variant) is less than about 1000 nucleotides long. In another embodiment, the polynucleotide is between about 4 nucleotides to about 925 nucleotides. More preferably, the polynucleotide is 925 nucleotides as shown in SEQ ID NO: 1.

In one embodiment of the present invention a nucleic acid molecule comprising or consisting of a sequence which is a variant of the P. pastoris MLS1 promoter has at least 70% identity to SEQ ID NO: 1. The nucleic acid sequence can preferably have at least 75% or 80% identity to the wild-type promoter. Even more preferably, the nucleic acid sequence can have 85%, 90%, 95%, 98%, 99%, 99.9% or even higher identity to the SEQ ID NO: 1.

In a preferred embodiment, the MLS1 5′ regulatory region disclosed herein, is approximately a 925 nucleotide fragment that can be operably linked to heterologous DNA coding sequences encoding at least one polypeptide. Suitable heterologous DNA coding sequences encoding at least one polypeptide which could be operably linked with the MLS1 5′ regulatory region for example, include but are not limited to, the kringle 3 (K3) domain of human plasminogen (Example 2). Any heterologous DNA coding sequence used with the present invention preferably contains a 5′ ATG start codon and a 3′ stop codon.

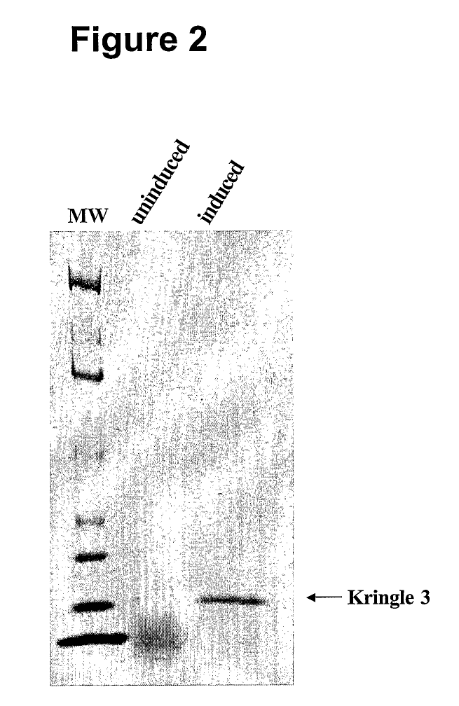

Additionally, a vector that contains a DNA fragment derived from the 5′ regulatory region of the MLS1 gene of P. pastoris, operably linked to the human K3 domain, is provided. The DNA fragment, corresponding to the promoter region of the MLS1 gene can regulate expression of the K3 domain in the host strain. An example of the K3 domain regulated by the MLS1 promoter in P. pastoris is shown in FIG. 2.

In another aspect of the present invention, the combination of the MLS1 promoter operably linked to a heterologous DNA coding sequence may be inserted into a suitable vector. Numerous useful vectors have been described for yeast transformation and are known to those skilled in the art. The vectors should contain necessary elements which render the vector capable of growth amplification and rapid propagation in bacteria or yeast (Higgins and Cregg, 1998, Pichia Protocols, Humana Press).

The heterologous DNA sequences that can be operably linked to the MLS1 5′ regulatory region include, but are not limited to, one of the following: erythropoietin, cytokines such as interferon-a, interferon-b, interferon-g, interferon-w, and granulocyte-CSF, GM-CSF, coagulation factors such as factor VIII, factor IX, and human protein C, antithrombin III, thrombin, soluble IgE receptor a-chain, IgG (e.g. a-CD20, a-CD33, a-TNF a, a-EGPR), IgG fragments, IgG fusions, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteo-protegerin, a-1-antitrypsin, a-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1, IL-2 receptor agonist, and the yeast alpha mating secretion domain alone, or fused to any heterologous sequence (Larouche, et al. 1994, Bio/Technology, 12: 1119-1124).

In another embodiment, suitable host cells for the induction of the MLS1 5′ regulatory region include yeast preferably from the genus Pichia. Yeasts belonging to the genus Pichia according to the present invention include for example, Pichia pastoris, Pichia finlandica, Pichia trehalophila, Pichia koclamae, Pichia membranaefaciens, Pichia methanolica, Pichia minuta (Ogatae minuta, Pichia lindneri), Pichia opuntiae, Pichia thermotolerans, Pichi salictaria, Pichia guercum, Pichia pijperi, Pichia stiptis, Pichia sp., and other yeasts, but not limited thereto. In yet another embodiment, suitable host cells for the induction of the Pichia MLS1 5′regulatory region include yeast such as Saccharomyces, Hansenula, Candida and Pichia. The introduction of the promoter region into the host strains by a compatible vector can be accomplished by suitable transformation techniques known to those skilled in the art. For the transformation of the pBK396 vector carrying the MLS1 promoter and the K3 domain, see Example 2.

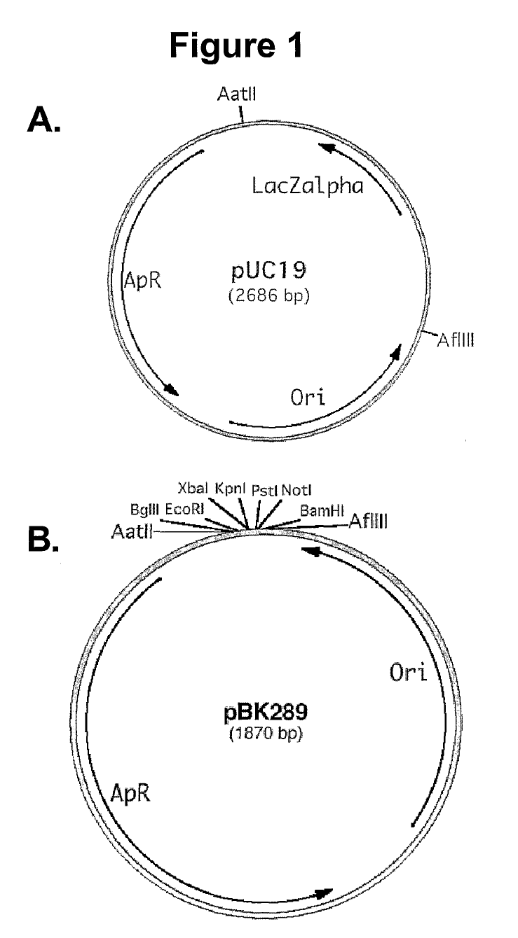

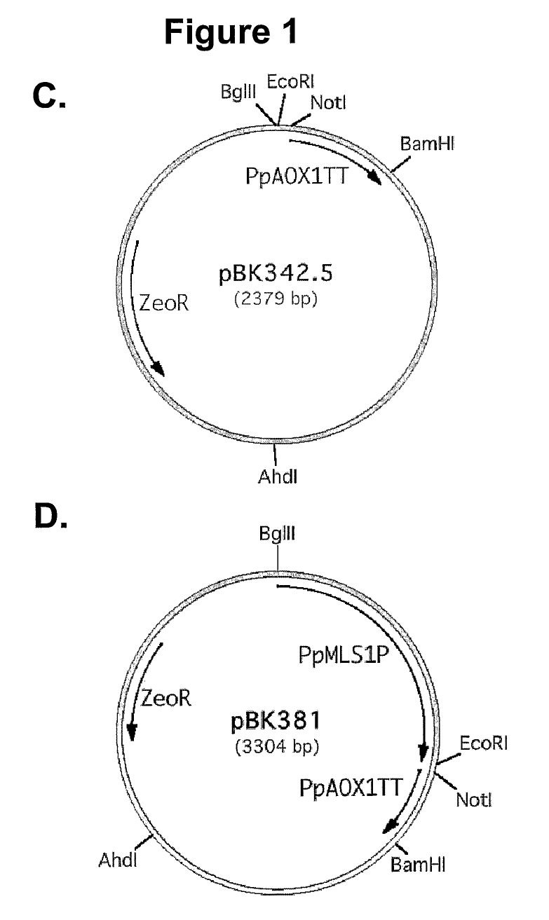

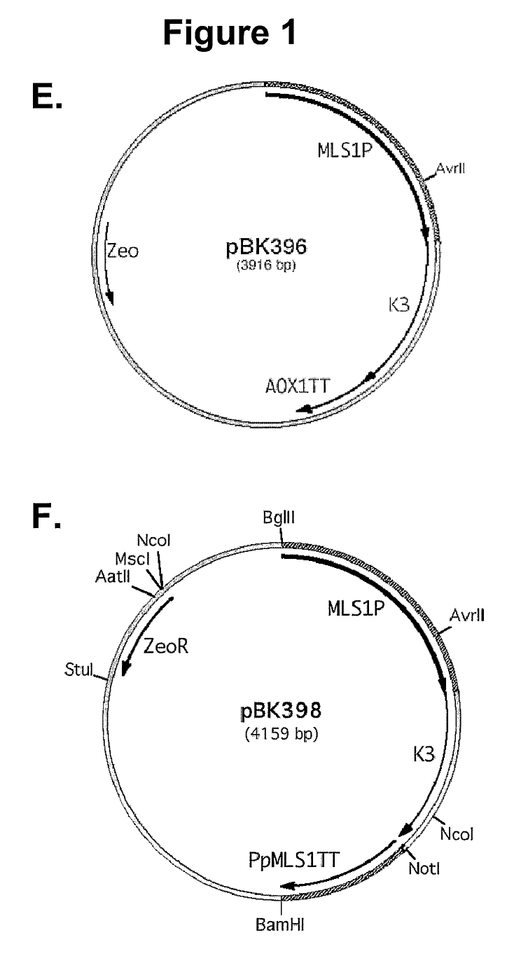

Additionally, the P. pastoris MLS1 3′ regulatory region encoding a transcriptional terminator can be used in the place of the AOX1 transcriptional terminator in pBK396. This MLS1 transcriptional terminator (SEQ ID NO: 4) was cloned from a wild type P. pastoris strain, NRRLY-11430 (American Type Culture Collection, ATCC) using a 5′ primer (PpMLS1-TT/UP) (SEQ ID NO: 5) and a 3′ primer (PpMLS1-TT/LP) (SEQ ID NO: 6) (Example 2). This terminator fragment was amplified by PCR and sublconed into a pCR2.1 vector. A BamHI/NotI terminator fragment was then subcloned from this vector into the BamHI/NotI sites in pBK396, resulting in pBK398 (FIG. 1F).

The following are examples which illustrate the compositions and methods of this invention. These examples should not be construed as limiting—the examples are included for the purposes of illustration only.

### EXAMPLE 1

### Isolation of the P. pastoris 5′ Regulatory Region of the MLS1 Gene

The MLS1 5′ regulatory promoter region was cloned from the wild type Pichia pastoris strain NRRLY-11430 (ATCC) using a 5′ PpMLS1-P/UP primer 5′ AGATCTTCCTCACAGGTAAGGTAGAATTACC 3′ (SEQ ID NO: 2) and a 3′ PpMLS1-P/LP primer 5′ GAATTCTTTTGTGATAAAGCGGTAA-ATCTGG 3′ (SEQ ID NO: 3). This 925 bp DNA fragment was subsequently sequenced using the same cloning primers confirming the correct MLS1 promoter sequence (SEQ ID NO: 1). The MLS1 promoter was then subcloned into an expression vector as detailed below, resulting in pBK396.

### EXAMPLE 2

### Construction of Plasmid pBK396 and pBK398 and Expression of Kringle 3 Under the Control of the MLS1 Promoter

An inventor-designed multiple cloning site (MCS) (SEQ ID NO: 7) was subcloned into the AatII/AflIII sites of the pUC19 vector (FIG. 1A) to produce vector pBK289 (FIG. 1B). A 257 bp Notl/BamH1 fragment containing the AOX1 transcription terminator sequence and a 1.2 kb AhdI/BglII zeocin resistance cassette (Invitrogen) was subcloned into pBK289, producing pBK342.5 (FIG. 1C). The 925 bp BglII/EcoRI fragment containing the 5′ regulatory region of the MLS1 gene was subcloned into the BglII/EcoRI sites of pBK342.5 to produce pBK381 (FIG. 1D).

The human plasminogen kringle 3 DNA sequence with 5′ BstBI and 3′ AccI sites filled in to provide blunt ends and was subcloned into the blunt-ended AfeI site of pBK381 producing pBK396 (FIG. 1E). A BamHI/Not1 fragment encoding the MLS1 3′ terminator region was subcloned into pBK396, replacing the AOX1 terminator, resulting in pBK398 (FIG. 1F). These vectors-pBK396 and pBK398—were linearized with AvrII (AvrII site in the MLS1 promoter region of the plasmid) prior to transformation by electroporation into the P. pastoris strain PBP33 (his4, arg4, ura3, ade1, och1::URA3, ade1::ADE1), resulting in strain BKY285 (see Example 3)

### EXAMPLE 3

### Transformation of pBK386 and pBK388 Vectors into P. pastoris Strain PBP33 Resulting in BKY285 and BKY285.1.

1. The vector DNA of pBK386 and pBK388 was prepared by adding sodium acetate to a final concentration of 0.3 M. One hundred percent ice cold ethanol was then added to a final concentration of 70% to the DNA sample. The DNA was pelleted by centrifugation (12000g×10 min) and washed twice with 70% ice cold ethanol. The DNA was dried and resuspended in 50 ml of 10 mM Tris, pH 8.0. The PBP33 yeast culture (supra) to be transformed was prepared by expanding a smaller culture in BMGY (buffered minimal glycerol: 100 mM potassium phosphate, pH 6.0; 1.34% yeast nitrogen base; 4×10⁻⁵% biotin; 1% glycerol) to an O.D. of ˜2-6. The yeast cells were then made electrocompetent by washing 3 times in 1M sorbitol and resuspending in ˜1-2 mls 1M sorbitol. Vector DNA (1-2 mg) was mixed with 100 ml of competent yeast and incubated on ice for 10 min. Yeast cells were then electroporated with a BTX Electrocell Manipulator 600 using the following parameters; 1.5 kV, 129 ohms, and 25 mF. One milliliter of YPDS (1% yeast extract, 2% peptone, 2% dextrose, 1M sorbitol) was added to the electroporated cells. Transformed yeast was subsequently plated on selective agar plates containing zeocin. pBK386 in PBP33 resulted in BKY285 and pBK388 in PBP33 resulted in BKY285.1.

Culturing Conditions for P. pastoris BKY285 and BKY285.1

After incubation on YPD medium containing 100 μg/ml zeocin, colonies were restreaked. Zeocin-resistant clones were selected and grown in buffered dextrose-complex medium (BMDY) media (1% yeast extract, 2% peptone, 100 mM potassium phosphate buffer (pH 6.0), 1.34% yeast nitrogen base, 4×10⁻⁵% biotin, and 2% glucose) for two days and then harvested and resuspended in buffered acetate induction media—BMAcY with 0.5% NaOAc for 1 day.

### EXAMPLE 4

### SDS-PAGE Analysis of Kringle 3 Expression Regulated by MLS1 Promoter and Terminator

Induced and uninduced cultures of Kringle 3 protein in strain BKY285 were analyzed by SDS-PAGE (FIG. 2). Appropriate volumes of sample loading buffer and culture media were subjected to sodium dodecyl sulfate-polyacrylamide gel electrophoresis (SDS-PAGE) with precast gels according to the manufacturer's instructions (NuPAGE bis-Tris electrophoresis system; Invitrogen Corporation, Carlsbad, Calif.). The gel proteins were stained with Coomassie brilliant blue stain (Bio-Rad, Hercules, Calif.).

SEQUENCE LISTING

| | |
|---|---|
| P. pastoris MLS1 promoter | SEQ ID NO: 1 |

tcctcacaggtaaggtagaattaccagaaagagaggaacaagaggcttgg
attcagaacagagtcaatct-gaagtctgacaccacctttttccacatga
ttccatacgaagaggctggtgaatatttccaggacttggtcaggctggct
gctgatcctgaattgacttttgacagcgacaaaatctacaacgctaccaa
agcaggccaagaactcaagtcgaaat-actgggcccgtaacaaagagatc
agaaaattgggaaccagtgcttaatccatacgtcgctggcattattaaac
taggttttgatgaaaactcagcatgtgattgagaactctcagttggttct
ttttcgttttcactttacgaattgttttagaaaggcatcatctaatgtaa
tctgtatacactttattgcattatatatactccaccttgctgttcatctc
gtcatcttcggattcttagctgctccatctgccccgggggtgtgaaatgt
ccgactctccgagcagacggttgagcctcgcgccgcatcaacggataag-
gcatggtagtgacccctctcaacagcgggccatacagttctccgcccacc
gtcgccgcaaggatcaccataaaaccttgct-gctcccggcgttcttggg
tacttgacggacggagaacagacaggtttcagacccccccggaagttgac
acctaggtcagt-tataattgcaagtcacactacctgcacatagtaaatt
gctctcacccggttgagatccgaccagttctgttgactctgtttgcttcc
atgactctgctccccttggccgactgataagcatgttcatcccatcggcc
attgccgtgactcgggaatgacacc-ccagcaaacgtatataaacccaca
atccccccagatttaccgctttatcacaaaa

| | |
|---|---|
| PpMLS1-P/UP primer f: | SEQ ID NO: 2 |

5′-agatcttcctcacaggtaaggtagattacc-3′

| | |
|---|---|
| PpMLS1-P/LP primer: | SEQ ID NO: 3 |

5′gaattcttttgtgataaagcggtaaatctgg-3′
P. pastoris MLS1 transcriptional terminator

SEQ ID NO: 4

gtttgttgtcttttataaatgtctttttatatatgcattcatgccttgac
agcattgtttggttaatttcgggtcaattgagttc-gaatatatcattcg
atgtaagtcttgcacgaatgactgtagcacaagatgatgctccacgatgg
aatcgattctggcgtaggggacgttcaaatctaccagcatctgtttgaca
aaattttgaagtctcctaagagagagattgtctactacagcaaggccgag
ttcattgtatttatcgccttcgaagtcacttttgttcagagatcgaactt
tacctattcttgttgccaactggtggactagcttcgagttgagtgtcttt
gattcttgcaattgttgatttatctttgctattttgctgtctttttctgt
ggattgttcctttaatgtgaatattagtgagtctttgtggtctatcattt
cttttagtctgcgaatttgctcgtctttatcatctgtctgtgtttcgtgt
tcgccttcttttatcgtcatagagctgtcatgagtacctggaggtgttaa
tagattggtggtggaaccgttatctccttcttcactcacatcgctgacca
gttctgctatacgctgtcccgggttgttatggctttttggttc

| | |
|---|---|
| PpMLS1-TT/UP primer F. | SEQ ID NO: 5 |

5′-gcggccgcgtttgttgtcttttataaatgtc-3′

| | |
|---|---|
| PpMLS1-TT/LP primer F. | SEQ ID NO: 6 |

5′-ggatccaacacgaaacacagacagatgataaagac-3′

| | |
|---|---|
| multiple cloning site (MCS) | SEQ ID NO: 7 |

5′-gacgtcagatctcggaattcagcgctggtacctctagactgcaggcg
gccgcgggatccatgt-3′

1. An isolated polynucleotide comprising of a nucleic acid sequence selected from the group consisting of:
(a) SEQ ID NO:1;
(b) the complement of SEQ ID NO:1;
(c) a nucleic acid sequence at least 70%, at least 75%, at least 80%, at least 85%, at least 90%, at least 95%, at least 98%, at least 99% identity to SEQ ID NO: 1; and
(d) a nucleic acid sequence that hybridizes under stringent conditions to SEQ ID NO:1.

2. An isolated polynucleotide comprising or consisting of a nucleic acid sequence selected from the group consisting of:
(a) SEQ ID NO:4;
(b) the complement of SEQ ID NO: 4;
(c) a nucleic acid sequence at least 65% at least 75%, at least 80%, at least 85%, at least 90%, at least 95%, at least 98%, at least 99% identity to SEQ ID NO: 4; and
(d) a nucleic acid sequence that hybridizes under stringent conditions to SEQ ID NO:4.

3-9. (canceled)

10. The isolated polynucleotide sequence of claim 1 wherein the polynucleotide is a regulatory region of the noncoding region of Pichia MLS1 gene.

11. The isolated polynucleotide sequence of claim 1 ligated at the 5′ end to a polynucleotide encoding a protein sequence of interest.

12. The isolated polynucleotide of claim 11 wherein the polynucleotide encoding the protein sequence of interest is operably linked at the 3′ end to the isolated polynucleotide sequence of claim 2.

13. A vector comprising the isolated polynucleotide of claim 1.

14. The vector of claim 13 further comprising the isolated polynucleotide of claim 2.

15. A host cell comprising the isolated polynucleotide of claim 1.

16. The host cell of claim 15 wherein the host cell is selected from the group consisting of: Pichia pastoris, Pichia finlandica, Pichia trehalophila, Pichia koclamae, Pichia membranaefaciens, Pichia methanolica, Pichia minuta (Ogataea minuta, Pichia lindneri), Pichia opuntiae, Pichia thermotolerans, Pichi salictaria, Pichia guercum, Pichia pijperi, Pichia stiptis and Pichia sp.

17. The host cell of claim 15 further comprising the isolated polynucleotide of claim 2.

18. The host cell of claim 17 wherein the host cell is selected from the group consisting of: Pichia pastoris, Pichia finlandica, Pichia trehalophila, Pichia koclamae, Pichia membranaefaciens, Pichia methanolica, Pichia minuta (Ogataea minuta, Pichia lindneri), Pichia opuntiae, Pichia thermotolerans, Pichi salictaria, Pichia guercum, Pichia pijperi, Pichia stiptis and Pichia sp.

19. A method for producing a heterologous protein in a host from the genus of Pichia comprising the steps of:
(a) introducing into the host the polynucleotide sequence of claim 1 operably linked to a polynucleotide encoding the heterologous protein; and
(b) culturing the host to produce the heterologous protein.

20. The method of claim 19, wherein the heterologous protein is selected from the group consisting of: erythropoietin, cytokines such as interferon-α, interferon-β, interferon-γ, interferon-ω, and granulocyte-CSF, GM-CSF, coagulation factors such as factor VIII, factor IX, and human protein C, antithrombin III, thrombin, soluble IgE receptor α-chain, IgGs, IgG fragments, IgG fusions, IgM, interleukins, urokinase, chymase, and urea trypsin inhibitor, TGF-binding protein, epidermal growth factor, growth hormone-releasing factor, annexin V fusion protein, angiostatin, vascular endothelial growth factor-2, myeloid progenitor inhibitory factor-1, osteoprotegerin, α-1-antitrypsin, α-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1, IL-2 receptor agonist and the yeast alpha mating secretion domain either alone, or fused to any heterologous sequence.

21. The method of claim 20 wherein the polynucleotide encoding the heterologous protein is further operably linked to the polynucleotide of claim 2.

22. The method of claim 21, wherein the heterologous protein is selected from the group consisting of: erythropoietin, cytokines such as interferon-α, interferon-β, interferon-γ, interferon-ω, and granulocyte-CSF, GM-CSF, coagulation factors such as factor VIII, factor IX, and human protein C, antithrombin III, thrombin, soluble IgE receptor α-chain, IgGs, IgG fragments, IgG fusions, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1-antitrypsin, α-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1, IL-2 receptor agonist and the yeast alpha mating secretion domain either alone, or fused to any heterologous sequence.

23. The isolated polynucleotide sequence of claim 2 wherein the polynucleotide is a regulatory region of the noncoding region of Pichia MLS1 gene.

24. The isolated polynucleotide sequence of claim 2 ligated at the 3′ end to a polynucleotide encoding a protein sequence of interest.

25. A vector comprising the isolated polynucleotide of claim 2.

26. A host cell comprising the isolated polynucleotide of claim 2.

27. The host cell of claim 26 wherein the host cell is selected from the group consisting of: Pichia pastoris, Pichia finlandica, Pichia trehalophila, Pichia koclamae, Pichia membranaefaciens, Pichia methanolica, Pichia minuta (Ogataea minuta, Pichia lindneri), Pichia opuntiae, Pichia thermotolerans, Pichi salictaria, Pichia guercum, Pichia pijperi, Pichia stiptis and Pichia sp.

28. A method for producing a heterologous protein in a host from the genus of Pichia comprising the steps of
(a) introducing into the host the polynucleotide sequence of claim 2 operably linked to a polynucleotide encoding the heterologous protein; and
(b) culturing the host to produce the heterologous protein.

29. The method of claim 28, wherein the heterologous protein is selected from the group consisting of: erythropoietin, cytokines such as interferon-α, interferon-β, interferon-γ, interferon-ω, and granulocyte-CSF, GM-CSF, coagulation factors such as factor VIII, factor IX, and human protein C, antithrombin III, thrombin, soluble IgE receptor α-chain, IgGs, IgG fragments, IgG fusions, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1-antitrypsin, α-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1, IL-2 receptor agonist and the yeast alpha mating secretion domain either alone, or fused to any heterologous sequence.
